(19) United States
(12) Patent Application Publication
(10) Pub. No.: US 2005/0008607 A1
(43) Pub. Date: Jan. 13, 2005

(54) Composition containing a carbonyl amine

(75) Inventors: Alexandre Cavezza, Tremblay-En-France (FR); Maria Dalko, Gif S/Yvette (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/856,819

(22) Filed: Jun. 1, 2004

Related U.S. Application Data
(60) Provisional application No. 60/479,160, filed on Jun. 18, 2003.

(30) Foreign Application Priority Data
Jun. 5, 2003 (FR) 0306798

(51) Int. Cl. A61K 31/198; A61K 7/075; A61K 7/08

(52) U.S. Cl. 424/70.22; 514/567

(57) ABSTRACT

The present invention relates to a composition suitable for topical application to the skin, containing, in a physiologically acceptable medium, at least one carbonyl amine of formula (I): 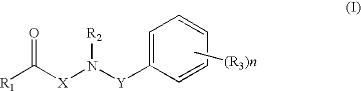
It also relates to the cosmetic use of this compound, especially for preventing or combating the signs of skin ageing, especially for relaxing the lines of the face and/or for smoothing out wrinkles, in particular expression wrinkles. The invention further relates to novel compounds of formula (II):

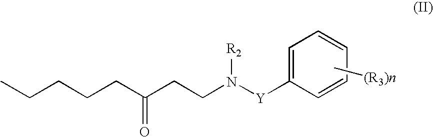

### REFERENCE TO PRIOR APPLICATIONS

This application claims priority to U.S. provisional application No. 60/479,160 filed Jun. 18, 2003, and to French patent application 0306798 filed Jun. 5, 2003, both incorporated herein by reference.

### FIELD OF THE INVENTION

The present invention relates to a composition suitable for topical application to the skin, comprising, in a physiologically acceptable medium, at least one carbonyl amine of given formula. It also relates to the amine itself, optionally in certain forms and environments, and the use of such an amine in a composition suitable for topical application to the skin, for example as an agent intended to relax the lines of the face and/or smooth out wrinkles, in particular expression wrinkles.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Women, and even men, currently have a tendency to wish to look youthful for as long as possible and consequently seek to fade out the signs of ageing on the skin, which are reflected in particular by wrinkles and fine lines. In this respect, the media and the fashion world report about products intended to keep the skin radiant and wrinkle-free for as long as possible, which are signs of youthful skin, and all the more so since physical appearance acts on the psyche and/or on the morale.

Up until now, wrinkles and fine lines were treated using cosmetic products containing active agents acting on the skin, for example by improving its cell renewal or alternatively by promoting the synthesis, or preventing the degradation, of collagen.

Although these treatments make it possible to act on the wrinkles and fine lines caused by chronological and/or actinic ageing, they have no effect on the expression wrinkles, which require an intervention on the contractile muscle component of the wrinkles present in the skin.

Up until now, the only means commonly used for acting on expression wrinkles is botulinum toxin, which is in particular injected into the wrinkles of the glabella, which are the wrinkles between the eyebrows (see J. D. Carruters et al., J. Dermatol. Surg. Oncol., 1992, 18, pp. 17-21).

There have also been proposed various compounds capable of affording a dermo-decontracting effect when they are applied topically to the skin, thus making it possible to act on expression wrinkles via another route. Among these compounds, mention may in particular be made of the calcium channel-associated receptor antagonists (FR-2 793 681), and in particular manganese and its salts (FR-2 809 005) and alverine (FR-2 798 590), and the chlorine channel-associated receptor agonists, including glycine (EP-0 704 210) and certain extracts of Iris pallida (FR-2 746 641).

### SUMMARY OF THE INVENTION

There is still a need for compounds that are effective in smoothing out or eliminating expression wrinkles. Now, the inventors have discovered, surprisingly, that certain carbonyl amines can satisfy this need.

These amines have already been described as intermediates in the synthesis of antihistamines (EP-0 209 158), of products intended for the treatment of Parkinson's disease (U.S. Pat. No. 3,009,915), of anti-inflammatory agents (WO 02/083624) and of anti-spasmodics (FAUST J. A. et al., J. Am. Chem. Soc. (1959), 81, 2214-19). It has also been suggested to use them to promote the absorption of substances in the digestive tract (EP-0 009 609) or as L-DOPA precursors in effervescent pharmaceutical compositions (CH-627 077). They have, moreover, been described as cationic surfactants for applications in the textile field (JP-30 003 216).

Amines corresponding to the definition of those which are the subject of the present invention have also been proposed for use as anti-spasmodics, intended for oral, parenteral or rectal administration, for the purpose of relaxing the smooth muscle of the gastrointestinal or urogenital tract or of the bronchial system (U.S. Pat. No. 3,996,245).

Along the same lines, application WO 92/04371 discloses kininogenase inhibitors which can be used in particular as smooth muscle anti-spasmodics, in the treatment of asthma or of angina, comprising compounds that can in particular be administered in the form of compositions applied to the skin.

These documents do not suggest that the compounds disclosed could have any effect on striated muscles, in particular on the muscles of the skin. Now, it is known in the literature that compounds which are effective on smooth muscle do not necessarily have the effect on striated muscle, and vice-versa (American Journal of Veterinary Research (1996), 57(10), 1497-1500; Planta Medica (1970), 18(3), 222-6; Nippon Yakurigaku Zasshi (1976), 72(1), 41-52; Drug Development Research (1992), 25(2), 161-9).

Thus, it was not obvious that the amines according to the present invention could have a relaxing effect on striated muscle, making possible their application in topical cosmetic or dermatological compositions, and in particular for anti-ageing purposes.

### DETAILED DESCRIPTION OF THE PREFERRED EMBODIMENTS

One subject of the present invention is therefore a composition suitable for topical application to the skin, comprising, in a physiologically acceptable medium, at least one compound of formula (I): 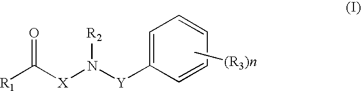
in which:
R₁ is a saturated or unsaturated, linear or branched C₁-C₁₀ alkyl group optionally substituted with an oxo group or with at least one of the groups: —OR, —SR, —NRR′, —COR, —COOR, —CO—NRR′, —NR—CO—R′, F and CF₃,
   where R and R′ independently denote a hydrogen atom or a linear or branched C₁-C₄ alkyl group;
R₂ denotes a hydrogen atom or a saturated or unsaturated, linear or branched C₁-C₈ alkyl group optionally substituted with an oxo group;
R₃ denotes, independently when more than one R₃ is present,
   a saturated or unsaturated, linear or branched C₁-C₆ alkyl group or an aryl group,
   a group —CN, —OR, —SR, —NRR′, —COR, —COOR, —CONRR′, —NR—CO—R′ or —CF₃, or
   a halogen atom,
      where R and R′ have the meaning given above;
X is a C₁-C₉ alkylene or alkenylene chain optionally substituted with an oxo group or with at least one of the groups: —OR, —SR, —NRR′, —COR, —COOR, —CO—NRR′, —NR—CO—R′ and CF₃ and/or with an aryl group, where R and R′ have the meaning given above;
Y is a C₁-C₂₀ alkylene or alkenylene chain optionally substituted with at least one of the groups: —OR, —SR, —NRR′, —COOR, —CO—NRR′, —NR—CO—R′, F and CF₃ and/or with an aryl group, where R and R′ have the meaning given above;
n is 0-5,
including its addition salts with an acid.

In formula (I), the alkyl groups can preferably be chosen, as appropriate, from the following groups: methyl, ethyl, n-propyl, isopropyl, n-butyl, isobutyl, tert-butyl, pentyl, hexyl, heptyl, octyl, nonyl, decyl, undecyl and dodecyl.

For its part, the aryl group can for example be selected from the group consisting of a benzyl group and a phenyl group.

The alkylene or alkenylene chain is preferably an ethylene, trimethylene, tetramethylene, pentamethylene, vinylene or propenylene chain.

The halogen atom preferably is a fluorine, chlorine or bromine atom.

As salts of the compound of formula (I), preferred salts include those obtained by addition of the compound of formula (I) with an inorganic acid, chosen in particular from hydrochloric acid, sulphuric acid, nitric acid and phosphoric acid, or with an organic acid, chosen in particular from malonic acid, succinic acid, fumaric acid, lactic acid, glycolic acid, citric acid and tartaric acid.

The compounds of formula (I) can in particular be prepared according to a process similar to that described in Example 1.

According to a preferred embodiment of the invention, the compound of formula (I) is such that at least one of the following conditions is satisfied:
R₁ is a saturated or unsaturated, linear or branched C₁-C₅ alkyl group,
R₂ is a hydrogen atom or a C₁-C₃ alkyl group,
R₃ is a saturated or unsaturated, linear or branched C₁-C₆ alkyl group,
n is equal to 0, 1 or 2,
X is a C₁-C₄ alkylene chain, preferably an ethylene chain,
Y is a C₁-C₅, preferably C₁-C₃, alkylene chain.

Even more preferably, two or more, including all, the above conditions are satisfied.

Examples of compounds of formula (I) which are useful in the present invention include the compounds having the following formulae:

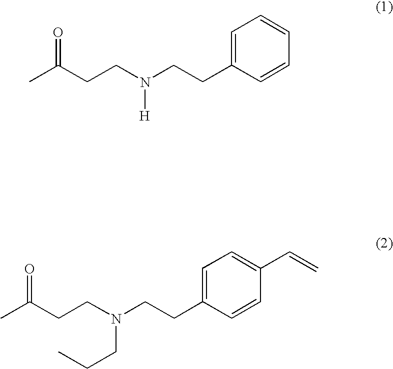

Other examples of compounds of formula (I) which are particularly useful in the present invention comprise those corresponding to general formula (II) below: 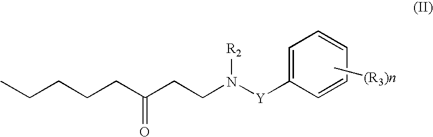
in which R₂, R₃, n and Y have the meaning given above for general formula (I).

To the inventor's knowledge, the compounds of formula (II) above are novel.

A subject of the invention is therefore also compounds corresponding to formula (II) 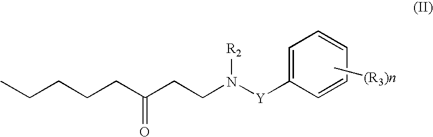
in which:
R₂ denotes a hydrogen atom or a saturated or unsaturated, linear or branched C₁-C₈ alkyl group optionally substituted with an oxo group;
R₃ independently denotes
   a saturated or unsaturated, linear, branched or cyclic C₁-C₆ alkyl group or an aryl group,
   a group —CN, —OR, —SR, —NRR′, —COR, —COOR, —CONRR′ or —CF₃, or
   a halogen atom,
   where R and R′ independently denote a hydrogen atom or a linear or branched C₁-C₄ alkyl group;
Y is a C₁-C₂₀ alkylene or alkenylene chain optionally substituted with at least one of the groups —OR, —SR, —NRR′, —COOR, —CO—NRR′, —NR—CO—R′, F and CF₃ and/or with an aryl group, where R and R′ have the meaning given above;
n is 0-5,
and its addition salts with an acid.

Advantageously, the compounds of formula (II) according to the invention are such that they satisfy at least one, including two or more, for example all, of the following conditions:
R₂ is a hydrogen atom or a saturated, linear C₁-C₈ alkyl group optionally substituted with an oxo group, preferably a C₁-C₃ alkyl group,
R₃ is a group —OR where R is a hydrogen atom or a linear or branched C₁-C₄ alkyl group,
n is equal to 0, 1 or 2, preferably 0,
Y is a C₁-C₅, preferably C₁-C₃, alkylene chain.

Examples of such compounds are in particular those corresponding to formulae (3) to (7) below:

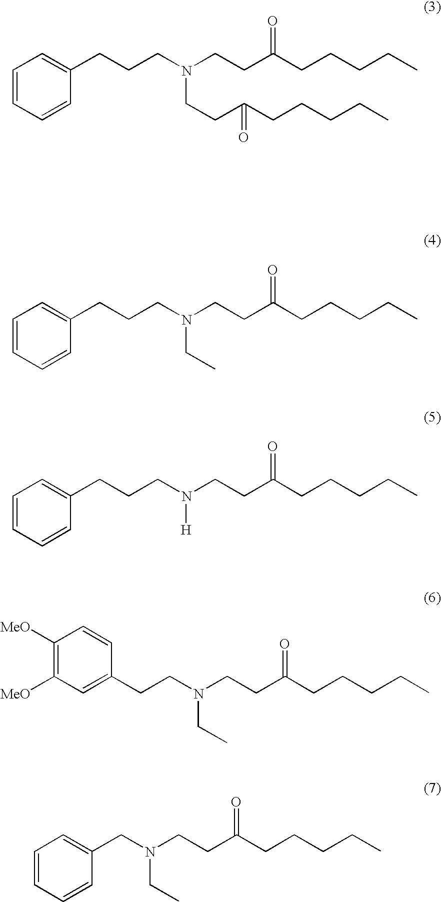

The amount of compound of formulae (I) and (II) which can be used according to the invention depends, of course, on the desired effect and can therefore vary within a broad range. Generally speaking the amount is an amount effective to provide a dermo-decontracting effect, and/or affect the contractile muscle component of the wrinkles present in the skin, and/or prevent or combat the signs of skin ageing, and/or to relax the lines of the face and/or to smooth out wrinkles, especially where the wrinkles are expression wrinkles. To give an order of magnitude, this compound can be used in an amount representing from 0.01% to 10% of the total weight of the composition, preferably in an amount representing from 0.05% to 5% of the total weight of the composition, more preferably in an amount representing from 0.1% to 2% of the total weight of the composition.

The composition according to the invention is suitable for topical application to the skin and it therefore contains a physiologically acceptable medium, i.e. a medium compatible with the skin and optionally with its integuments (eyelashes, nails, hair) and/or the mucous membranes.

The invention composition can be provided in any form including any and all forms used in the cosmetics and dermatology fields, and it can in particular be in the form of an optionally gelled solution, an optionally two-phase lotion-type dispersion, an emulsion obtained by dispersion of a fatty phase in an aqueous phase (O/W) or vice versa (W/O), or a triple emulsion (W/O/W or O/W/O) or a vesicular dispersion of ionic and/or nonionic type. These compositions can be prepared according to usual methods in view of this disclosure. According to this invention, use is preferably made of a composition in the form of an oil-in-water emulsion.

This composition may be more or less fluid and may have the appearance of a white or coloured cream, an ointment, a milk, a lotion, a serum, a paste or a foam. It can optionally be applied in the form of an aerosol. It can also be provided in solid form, in particular in the form of a stick. It can be used as a care product and/or as a make-up product for the skin.

In a known manner, the composition used according to the invention can also contain adjuvants useful in the cosmetics and dermatology fields, such as hydrophilic or lipophilic gelling agents, hydrophilic or lipophilic active agents, preserving agents, antioxidants, solvents, fragrances, fillers, screening agents, pigments, odour absorbers and dyestuffs. The amounts of these various adjuvants are those conventionally used in the field under consideration, and are, for example, from 0.01 to 20% of the total weight of the composition. Depending on their nature, these adjuvants can be introduced into the fatty phase, into the aqueous phase or in lipid vesicles. In any event, these adjuvants and the proportions thereof will preferably be chosen so as not to harm the desired properties of the compounds of formula (I) according to the invention.

When the composition used according to the invention is an emulsion, the proportion of the fatty phase can preferably range from 5 to 80% by weight, and more preferably from 5 to 50% by weight, relative to the total weight of the composition. The oils, the emulsifiers and the co-emulsifiers used in the composition in the form of an emulsion are selected from the group consisting of those conventionally used in the field under consideration. The emulsifier and the co-emulsifier are preferably present in the composition in a proportion ranging from 0.3 to 30% by weight, and more preferably from 0.5 to 20% by weight, relative to the total weight of the composition.

As oils which can be used in the invention, mention may be made of mineral oils (liquid petroleum jelly), oils of plant origin (avocado oil, soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (carnauba wax, ozokerite) can also be used as fatty substances.

As emulsifiers and co-emulsifiers which can be used in the invention, mention may be made, for example, of esters of a fatty acid and of polyethylene glycol, such as PEG-100 stearate, and esters of a fatty acid and of glycerol, such as glyceryl stearate.

As hydrophilic gelling agents, mention may in particular be made of carboxyvinyl polymers (carbomer), acrylic copolymers such as copolymers of acrylates/alkyl acrylates, polyacrylamides, polysaccharides, natural gums and clays, and, as lipophilic gelling agents, mention may be made of modified clays such as bentones, metal salts of fatty acids, hydrophobic silica and polyethylenes.

As active agents, the composition according to the invention can comprise at least one compound selected from the group consisting of: desquamating agents and/or moisturisers (such as α- and β-hydroxy acids, in particular 5-n-octanoylsalicylic acid, ceramides, hyaluronic acid, 2-oxothiazolidine-4-carboxylic acid, HEPES, honey and glycerol); depigmenting agents (including vitamin C and its derivatives such as ascorbyl glucoside); agents for stimulating fibroblast proliferation (including extracts of soybean); agents for stimulating keratinocyte proliferation (including retinoids such as retinol); agents for stimulating keratinocyte differentiation (including extracts of lupin and of maize); dermo-decontracting agents (such as adenosine, alverine and extracts of wild yam); tensioning agents (including colloidal silica, mixed silicates and acrylic-silicone latices); and anti-pollution agents or free-radical scavengers (including vitamin E and coenzyme Q10).

As UV-screening agents which can be used in the composition according to the invention, mention may be made of the following organic screening agents, cited, according to the case, using their CTFA or chemical name: ethylhexyl salicylate, ethylhexyl methoxycinnamate, octocrylene, phenylbenzimidazolesulphonic acid, benzophenone-3, benzophenone-4, benzophenone-5,4-methylbenzylidenecamphor, terephthalylidenedicamphorsulphonic acid, disodium phenyldibenzimidazoletetrasulphonate, 2,4,6-tris(diisobutyl-4′-aminobenzalmalonate)-s-triazine, anisotriazine, ethylhexyltriazone, diethylhexyl-butamidotriazone, methylenebis(benzotriazolyl)tetramethylbutylphenol, Drometrizole trisiloxane, 1,1-dicarboxy-(2,2′-dimethylpropyl)-4,4-diphenyl-butadiene, and mixtures thereof.

In addition to or in place of the organic screening agents above, the composition can contain inorganic photoprotective agents chosen, for example, from pigments or alternatively nanopigments (mean size of the primary particles: generally between 5 nm and 100 nm, preferably between 10 nm and 50 nm) of metal oxides such as, for example, nanopigments of titanium oxide (amorphous or crystallized in rutile and/or anatase form), of iron oxide, of zinc oxide, of zirconium oxide or of cerium oxide. These pigments may be uncoated or coated with alumina and/or aluminium stearate. Such coated or uncoated metal oxide nanopigments are in particular described in patent applications EP518772 and EP518773.

As will be demonstrated in the examples hereinafter, the applicant has demonstrated a dermo-decontracting effect of the compounds of formula (I) according to the invention, which makes it possible to envisage their use more particularly in smoothing out expression wrinkles.

A subject of the invention is therefore the cosmetic use of at least one compound of formula (I) or (II) as defined above, in a composition suitable for topical application to the skin, as an agent intended to prevent or combat the signs of skin ageing, in particular as an agent intended to relax the lines of the face and/or to smooth out wrinkles, in particular expression wrinkles.

A subject of the invention is also a cosmetic treatment process for the skin, especially for an aged skin, and more particularly for a wrinkled skin, comprising the topical application to said skin of a composition comprising, in a physiologically acceptable medium, at least one compound of formula (I) or (II) as defined above.

Given its dermo-decontracting properties, the composition according to the invention is advantageously intended to be applied to the areas of the face or of the forehead which are marked with expression wrinkles (these areas are included in areas of skin in need of the benefits of the invention), and/or on individuals with expression wrinkles. The wrinkles in question are preferably those lying radially around the mouth and/or the eyes, in particular the crow's-feet wrinkles, and/or lying on the forehead, in particular the “lion” wrinkle, located in the glabella, in between the eyebrows, and/or lying horizontally on the forehead.

The invention will now be illustrated with the following nonlimiting examples. In these examples, the amounts are indicated as percentages by weight.

### EXAMPLES

### Example 1

### Preparation of 1-(N-ethyl-(3-phenyl)propylamino)octan-3-one

The 1-(N-ethyl-(3-phenyl)propylamino)octan-3-one can be prepared according to a two-step process, according to the following scheme: 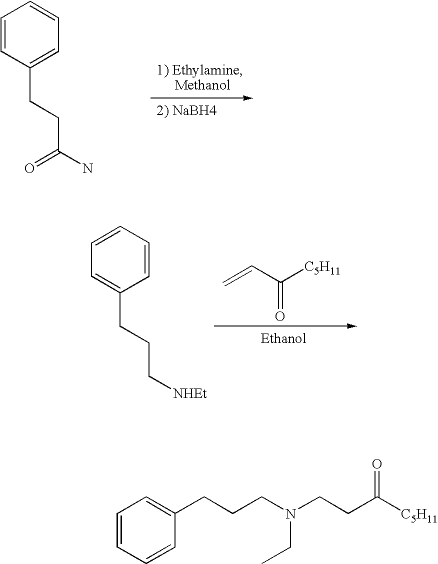
Step 1: Synthesis of N-ethyl-(3-phenyl)propylamine

2 g of 3-phenyl propionaldehyde are dissolved in 5 ml of methanol. 7.45 g of ethylamine in solution in ethanol (2 M) are added and the mixture is then allowed to react for 20 hours at ambient temperature. 0.68 g of sodium borohydride (1.2 eq.) is then added slowly and the mixture is left stirring for 20 hours at ambient temperature. The product is treated and purified on a silica column.

530 mg of N-ethyl-(3-phenyl)propylamine are obtained in the form of a product which appears as a single spot in thin layer chromatography (yield: 20%). The NMR and mass spectrometry results are in accordance with the expected structure.

Step 2: Synthesis of the 1-(N-ethyl-(3-phenyl)propylamino)octan-3-one

0.4 g of 1 -octene-3-one is dissolved in 5 ml of absolute ethanol. 0.49 g of the product resulting from step 1, in solution in 5 ml of absolute ethanol, is then added. The mixture is allowed to react for 15 hours at ambient temperature. The product is treated and purified on a silica column. 300 mg of product are obtained (yield: 30%). The NMR and mass spectrometry results are in accordance with the expected structure.

### Example 2

### Demonstration of the Dermo-Decontracting Effect of the Compounds According to the Invention

The compound of formula (4) was tested on a nerve-muscle coculture model which makes it possible to recreate a motor arc by innervating human striated muscle cells with explants of spinal cord and of spinal ganglia from rat embryos.

This test is able to predict an anti-wrinkle effect, as was demonstrated by the applicant in the case of diazepam, which inhibited muscle fibre contractions in this model, and the anti-wrinkle activity of which has been demonstrated in vivo.

a) Protocol

Human muscle cells derived from muscle cell samples from a normal donor are seeded in wells with a 1.8 cm² cross section (24-well culture dishes). After culturing for 10 days, these cells form a monolayer and fuse. At this stage, explants of spinal cord from 13-day rat embryos containing the spinal ganglia are deposited onto the culture.

Neurite growth out of the spinal cord explant is visible after culturing for one day. The first muscle fibre contractions are observed after co-culturing for 5 to 6 days and, after 3 weeks, all the muscle fibres in the vicinity of the explants contract.

The co-cultures are used after 21 days, when the muscle fibres are striated and have mature differentiated neuromuscular junctions.

A muscle fibre having regular contractions (at least 60 contractions per minute) is then selected in three different culture wells, and the number of contractions is counted over 30 seconds. The compound tested, diluted to 1/1000 in DMSO, is then incubated for 60 seconds in these wells, at a concentration of 100 μM. At the end of the incubation, the number of contractions is again counted over 30 seconds. The test is carried out in triplicate.

Botulinum toxin is used as a reference and tested at 0.1 and 1 μg/ml.

b) Results

Compound (4) according to the invention blocks the contractions of the abovementioned three muscle fibres at the concentration of 100 μM (0.3 μg/ml).

By comparison, botulinum toxin greatly decreases the frequency of the contractions after one minute and completely blocks the contractions after two hours of incubation, at the concentration of 1 μg/ml, and, at a concentration of 0.1 μg/ml, it has a blocking effect which is much weaker at one minute, but which is also complete after two hours.

Thus, the compounds according to the invention are dermo-decontracting agents which can be used to relax the lines of the face and to smooth out expression wrinkles.

### Example 3

### Cosmetic Composition

This composition is prepared in a manner which is conventional for those skilled in the art. The amounts indicated are in percentages by weight.

| | |
|---|---|
| Compound of formula (4) | 1% |
| Propylene glycol isostearate | 13% |
| Polyethylene glycol (8 EO) | 5% |
| Propylene glycol | 3% |
| Pentylene glycol | 3% |
| Glyceryl stearate and polyethylene glycol | 5% |
| (100 EO) stearate | |
| Oxyethylenated (20 EO) sorbitan | 0.5% |
| monostearate | |
| Oxyethylenated (20 EO) oxypropylenated | 1% |
| (5 PO) cetyl alcohol | |
| Gelling agents | 0.5% |
| C₁₂₋₁₅ alkyl benzoates | 4% |
| Ethanol | 3% |
| Sodium hydroxide | 0.12% |
| Preserving agents | 0.7% |
| Water | qs 100% |

This fluid is intended to be used in once-daily or twice-daily applications to the face and the forehead in order to reduce expression wrinkles.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition suitable for topical application to the skin, comprising,
a physiologically acceptable medium, and
at least one compound of formula (I): 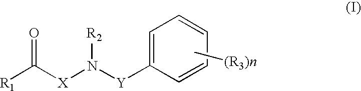
in which:
R₁ is a saturated or unsaturated, linear or branched C₁-C₁₀ alkyl group optionally substituted with an oxo group or with at least one of the groups: —OR, —SR, —NRR′, —COR, —COOR, —CO—NRR′, —NR—CO—R′, F and CF₃,
where R and R′ independently denote a hydrogen atom or a linear or branched C₁-C₄ alkyl group;
R₂ denotes a hydrogen atom or a saturated or unsaturated, linear or branched C₁-C₈ alkyl group optionally substituted with an oxo group;
R₃ independently denotes
a saturated or unsaturated, linear or branched C₁-C₆ alkyl group or an aryl group,
a group —CN, —OR, —SR, —NRR′, —COR, —COOR, —CONRR′, —NR—CO—R′ or —CF₃, or
a halogen atom,
where R and R′ have the meaning given above;
X is a C₁-C₉ alkylene or alkenylene chain optionally substituted with an oxo group or with at least one of the groups: —OR, —SR, —NRR′, —COR, —COOR, —CO—NRR′, —NR—CO—R′ and CF₃ and/or with an aryl group, where R and R′ have the meaning given above;
Y is a C₁-C₂₀ alkylene or alkenylene chain optionally substituted with at least one of the groups: —OR, —SR, —NRR′, —COOR, —CO—NRR′, —NR—CO—R′ and CF₃ and/or with an aryl group, where R and R′ have the meaning given above;
n is 0-5,
and its addition salts with acids.

2. A composition according to claim 1, wherein the compound of formula (I) is such that at least one of the following conditions is satisfied:
R₁ is a saturated or unsaturated, linear or branched C₁-C₅ alkyl group,
R₂ is a hydrogen atom or a C₁-C₃ alkyl group,
R₃ is a saturated or unsaturated, linear or branched C₁-C₆ alkyl group,
n is equal to 0 or 1,
X is a C₁-C₄ alkylene chain,
Y is a C₁-C₅, alkylene chain.

3. A composition according to claim 2, wherein the compound of formula (I) is such that X is an ethylene chain.

4. A composition according to claim 2, wherein the compound of formula (I) is such that Y is a C₁-C₃ chain.

5. A composition according to claim 1, comprising at least one of the following compounds:

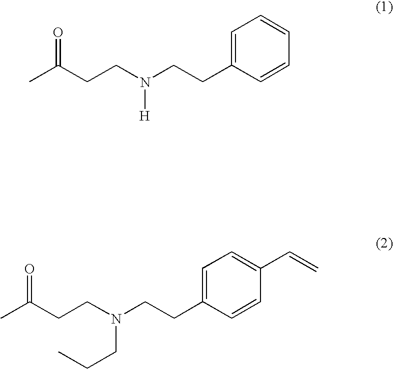

6. A composition according to claim 1, wherein the compound of formula (I) is present in an amount of 0.1% to 2% of the total weight of the composition.

7. A composition according to claim 1, comprising a dermo-decontracting effective amount of the at least one compound of formula (I), and wherein said composition is in the form of a gelled solution, a two-phase lotion-type dispersion, an O/W emulsion, a W/O emulsion, a W/O/W or O/W/O triple emulsion, or an ionic and/or non-ionic vesicular dispersion.

8. A compound corresponding to formula (II): 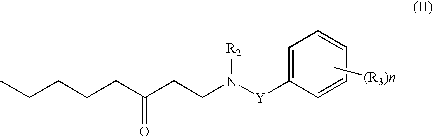
in which:
R₂ denotes a hydrogen atom or a saturated or unsaturated, linear or branched C₁-C8 alkyl group optionally substituted with an oxo group;
R₃ denotes
a saturated or unsaturated, linear, branched or cyclic C₁-C₆ alkyl group or an aryl group,
a group —CN, —OR, —SR, —NRR′, —COR, —COOR, —CONRR′ or —CF₃, or
a halogen atom,
where R and R′ independently denote a hydrogen atom or a linear or branched C₁-C₄ alkyl group;
Y is a C₁-C₂₀ alkylene or alkenylene chain optionally substituted with at least one of the groups —OR, —SR, —NRR′, —COOR, —CO—NRR′, —NR—CO—R′, F and CF₃ and/or with an aryl group, where R and R′ have the meaning given above;
n is 0-5,
and its addition salts with acids.

9. A compound according to claim 8, wherein at least one of the following conditions is satisfied:
R₂ is a hydrogen atom or a saturated, linear C₁-C₈ alkyl group optionally substituted with an oxo group,
R₃ is a group —OR where R is a hydrogen atom or a linear or branched C₁-C₄ alkyl group,
n is equal to 0, 1 or 2, preferably 0,
Y is a C₁-C₅, preferably C₁-C₃, alkylene chain.

10. A compound according to claim 9, wherein R₂ is a C₁-C₃ alkyl group.

11. A compound according to claim 9, wherein n is equal to 0.

12. A compound according to claim 9, wherein Y is a C₁-C₃ alkylene chain.

13. A compound according to claim 8, having one of the formulae below:

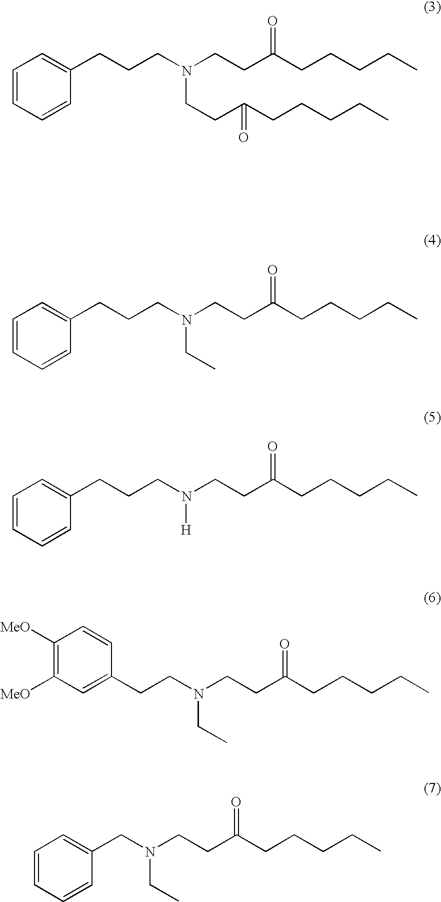

14. A method of preventing or combating signs of skin ageing, comprising applying the composition of claim 1 to skin in need thereof.

15. The method according to claim 14, wherein said at least one compound is present in an amount effective to relax the lines of the face and/or to smooth out wrinkles.

16. The method according to claim 14, wherein said at least one compound is present in an amount effective to treat expression wrinkles, and said composition is applied to expression wrinkles.

17. The method according to claim 15, wherein said composition is applied to the areas of the face and/or of the forehead which are marked with expression wrinkles.

18. The method according to claim 15, wherein said composition is applied to the wrinkles lying radially around the mouth and/or the eyes and/or horizontally on the forehead and/or located in between the eyebrows.

19. A method of preventing or combating signs of skin ageing, comprising applying the composition of claim 7 to skin in need thereof.

20. A method of preventing or combating signs of skin ageing, comprising applying the compound of claim 8 to skin in need thereof.

21. The method according to claim 20, wherein said compound is present in an amount effective to relax the lines of the face and/or to smooth out wrinkles.

22. The method according to claim 20, wherein said compound is present in an amount effective to treat expression wrinkles, and said composition is applied to areas of the face and/or of the forehead which are marked with expression wrinkles.

23. The method according to claim 20, wherein said composition is applied to the wrinkles lying radially around the mouth and/or the eyes and/or horizontally on the forehead and/or located in between the eyebrows.

24. A method of preventing or combating signs of skin ageing, comprising applying a dermo-decontracting effective amount of the compound of claim 8 to skin in need thereof.
